(19) United States
(12) Patent Application Publication
(10) Pub. No.: US 2007/0154418 A1
(43) Pub. Date: Jul. 5, 2007

(54) Method of treating skin requiring radiofrequency procedure

(76) Inventors: Judy Hattendorf, Marina Del Ray, CA (US); Steve Carlson, San Mateo, CA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/647,708

(22) Filed: Dec. 29, 2006

Related U.S. Application Data
(60) Provisional application No. 60/755,300, filed on Dec. 30, 2005.

(51) Int. Cl. A61K 8/31; A61N 1/39

(52) U.S. Cl. 424/59; 424/401; 607/1

(57) ABSTRACT

A treatment regimen for treating skin subject to radiofrequency procedures involves the application of supplemental composition(s) such as preparatory composition(s), protective composition(s), and combinations thereof, and a corrective composition.

### CROSS REFERENCE TO RELATED APPLICATION

This Application claims priority benefit of U.S. Provisional Application No. 60/755,300 filed Dec. 30, 2005 the entire disclosure of which is incorporated herein by this reference.

### BACKGROUND

1. Technical Field

This disclosure relates to the sequential topical application of treatment compositions in a prescribed fashion contributing to non-ablative cosmetic techniques. The disclosure further relates to the pre and post application of corrective compositions and one or more supplementary compositions to skin requiring a radiofrequency procedure. Kits for pre-treatment and post-treatment of radiofrequency procedures containing corrective compositions and supplementary compositions are also described.

2. Background of Related Art

Radiofrequency energy cosmetic techniques are used clinically for cosmetic and therapeutic purposes. Such techniques deliver radiofrequency energy into dermal tissues, thus contact the lower layers of skin with heat while leaving the epidermis unharmed and intact. Such heating can cause collagen to denature and fibrils to contract and thicken causing skin tightening. Furthermore, heating can cause wound-healing responses which produce additional skin tightening. Although state-of-the-art radiofrequency cosmetic therapies and procedures have been successful in treating patients in the appearance of, among other things, deep wrinkles, surface wrinkles, and skin discoloration, while minimizing detrimental conditions caused by ablative techniques, these therapies and procedures are problematic in that they can be painful and require localized anesthesia. Furthermore, radiofrequency procedures produce only subtle cosmetic results requiring repeat treatments in order to obtain the desired benefit. Thus patients are required to withstand painful repeat procedures in order to obtain significant cosmetic results. Moreover, these procedures are problematic in that scabbing and edema can occur after the radiofrequency procedure, which can take up to six months to subside.

Accordingly, there remains room for improvement in performing radiofrequency cosmetic techniques, and especially to minimize, reduce, or eliminate pain during the procedure, scarring, edema, and/or the need for multiple treatments after a radiofrequency procedure to obtain a cosmetically noticeable outcome.

### SUMMARY

Skin requiring a radiofrequency procedure is pre-treated in accordance with the present disclosure by preconditioning skin by the sequential topical application of one or more corrective compositions, and one or more supplementary compositions in a morning regimen; followed by the sequential topical application of one or more corrective compositions, including tretinoin, and one or more supplementary compositions in an evening regimen. Such preconditioning by the sequential application of such compositions can contribute to the benefits of the radiofrequency procedure by extending the duration of the cosmetic benefit received, as well as decreasing the frequency of repeated radiofrequency treatments to obtain the desired cosmetic benefit.

Optionally, after treating the preconditioned skin with a radiofrequency procedure, the skin can be post-treated by another sequential topical application of one or more corrective compositions, and one or more supplementary compositions in a morning regimen; followed by the sequential topical application of one or more corrective compositions, including tretinoin, and one or more supplementary compositions in an evening regimen. Post-treatment of preconditioned skin enhances the benefits of the radiofrequency procedure by extending the duration of the cosmetic benefit received, as well as reducing or eliminating adverse events such as reactions or complications like post-inflammatory hyperpigmentation, erythema (redness), edema and scarring. Moreover, post-conditioning treated skin can reduce the overall number of procedures needed in order to obtain a noticeable cosmetic benefit, minimizing pain associated with the process.

In addition, dermatological treatment regimens in accordance with the present disclosure can improve characteristics of a user's skin. The regimens include the application of one or more corrective compositions and the application of one or more supplementary compositions. Suitable corrective compositions include, for example, compositions which help to repair damage to the deeper layers of skin, or stable corrective compositions which contain one or more active ingredients sensitive to oxidation that remain stable for three years at room temperature. Suitable supplementary compositions include, for example: preparatory compositions which make skin more receptive to the corrective compositions; or protective compositions which further protect skin against damage from harmful UVA and UVB rays, as well as protecting the sensitive eye area. Depending on the nature of the one or more supplementary compositions, they can be applied before, after, or both before and after application of the corrective composition.

In a related aspect, the present disclosure is directed towards stable corrective compositions, wherein the active ingredient remains stable for three years at room temperature.

In another related aspect, the present disclosure is directed towards kits for pre-treating and post-treating skin subject to a radiofrequency procedure containing both one or more stable corrective compositions and one or more supplementary compositions.

These and other aspects of this disclosure will be evident upon reference to the following detailed description.

### DETAILED DESCRIPTION OF PREFERRED EMBODIMENTS

Treatment regimens in accordance with this disclosure include the sequential steps of: pre-treating the surface of skin in need of a radiofrequency procedure; performing a radiofrequency procedure; and optionally post-treating the skin, wherein the pre-treatment and post-treatment can include the sequential topical application of treatment compositions in a prescribed fashion.

The first step of the present method is pre-treatment. The pre-treatment step of the treatment regimen of the present disclosure is designed for pre-conditioning the skin to rendering the area of skin to be treated more receptive and responsive to a radiofrequency cosmetic procedure. For example, skin that is oily or dirty can be preconditioned to a healthier more hygienic state. It has been found that preconditioning skin by cleaning, toning, moisturizing, exfoliating, and/or using corrective compositions in accordance with the present disclosure can contribute to the benefits of a radiofrequency procedure by extending the duration of the aesthetic benefit received, as well as decreasing the frequency of repeated radiofrequency treatments to obtain the desired cosmetic benefit. Moreover, the sequential topical application of treatment compositions in accordance with the present disclosure contributes to radiofrequency cosmetic procedures by providing excellent aesthetic results with reduced recovery periods, while markedly improving rejuvenation goals and aesthetic expectations.

Thus, skin in need of radiofrequency treatment is pre-treated by preconditioning skin by the sequential topical application of one or more corrective compositions, and one or more supplementary compositions in a morning regimen; followed by the sequential topical application of one or more corrective compositions, including tretinoin, and one or more supplementary compositions in an evening regimen.

As used herein the word “treat,” “treating” or “treatment” refers to using the compositions of the present disclosure prophylactically to prevent outbreaks of undesirable dermatological symptoms, or therapeutically to ameliorate an existing undesirable dermatological condition, and/or extend the duration of the aesthetic benefit of a radiofrequency procedure, or reduce the frequency of repeated radiofrequency procedures.

Pre-treatment regimens in accordance with the present disclosure improve skin characteristics through sequential application of pre-selected skin care compositions to the skin of a user prior to the radiofrequency procedure. As used herein the word “corrective composition” refers to using the compositions of the present disclosure that have an active ingredient for treating any undesirable dermatological condition. Application of the corrective composition in combination with the one or more supplementary compositions provides improved effectiveness of the corrective composition compared to application of the corrective composition alone. As used herein the word “supplementary composition” refers to using compositions of the present disclosure which do not have active ingredient for treating undesirable dermatological conditions, however when used in conjunction with a corrective composition produce a beneficial effect.

The supplementary composition can be, for example, a preparatory composition that makes the skin of the user more receptive to the corrective composition. Alternatively, the supplementary composition can be a protective composition which protects skin against damage from harmful UVA and UVB rays.

Depending on the exact nature of the supplementary compositions employed, the supplementary composition can be applied before or after application of the corrective composition. For example, where the supplementary composition is a preparatory composition, the supplementary composition is applied before the corrective composition. Where the supplementary composition is a protective composition, the supplementary composition is applied after the corrective composition. In particularly useful embodiments, one or more supplementary compositions can advantageously be applied both before and after application of the corrective composition.

In embodiments, pre-treatment regimens in accordance with the present disclosure include a first treatment, such as in the morning hours, and a second treatment, such as in the evening hours. Both the first and second treatments include the topical application of one or more corrective compositions, along with one or more supplemental compositions. It should, of course be understood that the supplementary compositions used in the first treatment can be different from the supplementary compositions applied in the second treatment. Likewise, it should be understood that the corrective compositions used in the first treatment can be different from the corrective compositions applied in the second treatment.

In embodiments, the first treatment occurs in the morning hours and includes the application of an effective amount of one or more preparatory compositions (e.g., foaming gel, and toner) followed by application of an effective amount of one or more corrective compositions (e.g., exfoliator, and/or one or more hydroquinone compositions) followed by the application of an effective amount of one or more protective compositions (e.g., Obagi Healthy Skin Protection (daily sun protector)). In this embodiment, the second treatment occurs in the evening hours and includes the topical application of an effective amount of one or more preparatory compositions (e.g., foaming gel, and toner) followed by application of an effective amount of one or more corrective compositions (e.g., hydroquinone and tretinoin compositions). In embodiments, tretinoin is used either alone or in combination with other corrective compositions during the evening treatment.

Materials suitable for use as pre-treatment composition include corrective compositions and supplementary compositions pre-selected to clean, tone, moisturize, exfoliate, treat or precondition skin in need of a radiofrequency procedure. Non-limiting examples of pre-treatment compositions are listed below and include supplementary compositions such as cleanser compositions, toner compositions, and exfoliant compositions. Other suitable pre-treatment compositions include corrective compositions such as stable corrective compositions and stimulating corrective compositions. The pre-treatment compositions are categorized in various classes however this classification is not intended to limit the pre-treatment compositions in any way to only to those pre-treatment compositions belonging to the categories herein mentioned. Moreover, as described below, the same or different pre-treatment compositions can be used as post-treatment compositions in accordance with the present disclosure.

Supplementary Compositions

Non-limiting examples of supplementary compositions which may be combined with the corrective compositions of this disclosure are listed below.

Suitable supplementary compositions are categorized in various classes (e.g. preparatory compositions and protective compositions) however this classification is not intended to limit the supplemental compositions in any way to only those compositions belonging to the categories herein mentioned.

Preparatory Compositions

Throughout the pre-treatment regimen of the present disclosure, skin improvement may be slowed or worsened by skin becoming dirty or oily throughout the day and night. Dirt and oil clog pores and slow the corrective compositions from contacting the inner layers of skin. One class of supplementary compositions that may be combined with the corrective compositions of the present disclosure is preparatory compositions that make skin more receptive to the corrective step.

Thus, the pre-treatment regiment of the present disclosure includes the step of preparing skin to make it more receptive to the corrective step by applying preparatory compositions. Suitable preparatory compositions include cleansers, foaming gels, toners, and combinations thereof, which may be applied to the skin in the morning or evening portion of the treatment regimen.

The cleanser is applied to skin in amounts that provide the benefit to the skin of the user, such as in an amount sufficient to remove dirt and oil from the skin. Generally, the cleansers are soap-free and include water, detergent, surfactant, humectants, skin conditioning agent, PH adjustor, extracts, preservatives, fragrance and colorant, however, any cleaner suitable for removing dirt and oil from skin may be used. One commercially available cleanser is Obagi Nu-Derm® gentle cleanser available from OMP, Inc. of Long Beach, Calif. The Obagi Nu-Derm® gentle cleanser contains a combination of water, cocamidopropyl betaine, sodium lauroyl oat amino acids, sodium laureth sulfate, glycerin, aloe barbadensis gel, glycerth-7, apricot triethanolamine, sage extract, borage extract, phenoxythanol, methylparaben, propylparaben, ethylparaben, butylparaben, saponins, fragrance, and colorant.

Optionally, a foaming gel may be applied as one of the preparatory compositions in amounts that provide the benefit to the skin of the user, such as in an amount sufficient to remove dirt, oil and/or impurities to clean skin and leave it more receptive to treatment. Generally, foaming gels include water, detergent, surfactant, humectants, skin conditioning agent, PH adjustor, extracts, preservatives, fragrance and colorant, however any foaming gel may be applied that cleans the skin by removing dirt and/or oil. One commercially available foaming gel is Obagi Nu-Derm® foaming gel available from OMP, Inc. of Long Beach, Calif. The Obagi Nu-Derm® foaming gel contains a combination of water, sodium lauryl oat amino acids, cocamidopropyl betaine, sodium laureth sulfate, aloe barbadensis gel, alfalfa extract, borage extract, sodium chloride, xantham gum, saponins, phenoxyethanol, methylparaben, propylparaben, ethylparaben, butylparaben, fragrance and colorant.

Optionally, toner may be applied as a preparatory composition in amounts that provide the benefit to the skin of the user, such as in an amount sufficient to hydrate and tone skin while reducing the pH. Toner helps remove dirt, oils, and grime without overly drying out sensitive skin. Generally, toners include water, skin conditioner, astringent, minerals, moistening agent, vitamins and complexes thereof, anti-microbial, cleanser, extract, surfactant, anti-irritant, fragrance and colorant; however any commercially available skin toner may be used. One commercially available toner is Obagi Nu-Derm® toner available from OMP, Inc. of Long Beach, Calif. The Obagi Nu-Derm® toner contains a combination of water, aloe barbadensis gel, witch hazel distillate, potassium alum, sodium PCA, panthenol, DMDM hydantion, polysorbate 80, allantoin, sage extract, calendula officinalis extract, saponins, fragrance, and colorant.

During the treatment regimen, the preparatory composition(s) can advantageously be applied to damp skin of the face and neck with moistened fingertips. The face and neck of the user can be rinsed with warm water after application of the preparatory composition(s).

Protective Compositions

Skin improvement may be slowed or worsened by sunrays which may cause pigmentation and dryness. Accordingly, protective compositions are one class of supplementary compositions that optionally may be combined with the corrective compositions in the treatment regimens of the present disclosure to alleviate sun damage or dryness.

Suitable protective compositions include any composition capable of reducing skin damage, darkening, or dryness. In embodiments, protective compositions include sun block to help guard against photoaging by screening out ultraviolet light rays. In embodiments, suitable protective compositions include eye creams specially formulated to help control dryness.

One suitable commercially available sun block is Obagi Nu-Derm® Sunblock, from OMP, Inc. of Long Beach, Calif. formulated to prevent sunburn from UVB radiation and protect against long UVA rays linked to premature skin-aging. This protective composition is a daily sunscreen that provides broad-spectrum sun protection and can advantageously be applied every morning as part of a treatment regimen in accordance with this disclosure. The formulation is made of octinoxate, zinc oxide, butylparaben, cetearyl alcohol, citric acid, C13-14 isoparaffin, diethanolamine cetyl phosphate, disodium edetate, ethylparaben, isobutylparaben, isopropyl palmitate, laureth-7, methylparaben, octyl stearate, phenoxyethanol, polyacrylamide, polyether-1, polysorbate 60, propylparaben, purified water, sodium hydroxide, and triethoxycaprylylsilane.

Another suitable commercially available protective composition is Obagi Nu-Derm® Physical UV Block SPF 32. This composition contains zinc oxide USP, beeswax, butylene glycol, cetyl dimethicone, cetyl PEG/PPG-10/1 dimethicone, dimethicone, disodium EDTA, glycereth-26, hydrogenated castor oil, isopropyl palmitate, methylparaben, octyl stearate, propylparaben, purified water, sodium propylparaben, purified water, sodium chloride, triethoxycaprlylsilane, tocopherol acetate, and willowherb extract.

Another suitable commercially available protective composition is Obagi Nu-Derm® Healthy Skin Protection (SPF 35). This daily sunscreen uses active ingredients such as oxtinoxate and zinc oxide to provide broad spectrum sun protection.

Another suitable commercially available protective composition is Obagi Nu-Derm® Eye Cream. This composition is a light moisturizing cream especially suited for treating dry skin around the eye area. However, any skin moisturizer may be applied to the skin as needed.

Corrective Compositions

Non-limiting examples of corrective compositions which may be combined with the supplementary compositions of this disclosure are listed below.

Suitable corrective compositions are categorized in various classes (e.g. stimulating corrective compositions and stable protective compositions) however this classification is not intended to limit the corrective compositions in any way to only those compositions belonging to the categories herein mentioned. In fact, where necessary all ingredients used in the stable corrective compositions may be utilized to make the stimulating corrective compositions regardless of stability achieved.

Stimulating Corrective Compositions

Throughout the treatment regimen of the present disclosure, skin may be, among other things, regenerated by contacting the skin with one or more stimulating corrective compositions. Such compounds include those capable of the gradual bleaching of hyper-pigmented skin conditions such as chloasma melasma, freckles, sensile lentigines, and other unwanted areas of melanin hyper-pigmentation. Thus, suitable stimulating corrective compositions include, but are not limited to compositions having one or more active ingredients which help to repair damage to the deeper layers of skin, such as blenders, tretinoin compositions, sunfader, retin-A compositions, and combinations thereof.

The corrective compositions can be applied to the skin in amounts that provide the benefit to the skin of the user, such as in an amount sufficient to repair damage to the deeper layers of skin. Typically corrective compositions are applied to the skin in the two treatments per day, such as a morning (a.m.) and evening (p.m.) treatment. It should of course be understood that applying corrective compositions in one treatment is also possible, especially where the active ingredient is potent, such as tretinoin.

One corrective composition is a blender which promotes pigmentation correction at the cellular level promoting even skin color. Generally blenders include skin lightening agent such as hydroquinone, preservative, chelating agent, emulsifier, humectant, pH adjuster, antioxidant, emollient, reducing agent and water. Moreover, blenders with improved stability as those described below may also be used for correcting the skin in accordance with this disclosure.

One commercially available blender is Obagi Nu-Derm® Blender available from OMP, Inc. of Long Beach, Calif. The Obagi Nu-Derm® Blender contains a combination of hydroquinone USP 40mg/gm in a base of purified water, glycerin, cetyl alcohol, PPG-2 myristyl ether propionate, sodium lauryl sulfate, TEA-salicylate, lactic acid, phenyl trimethicone, tocopheryl acetate, sodiummetabisulfite, ascorbic acid, methylparaben, saponins, disodium EDTA, BHT and propylparaben.

Other suitable corrective compositions include retinoid containing compositions applied in amounts sufficient to provide benefit to the skin, such as medically prescribed tretinoin. Tretinoin skin preparations are a family of drugs all similar to Vitamin A available in gel or cream form. Tretinoin can advantageously be used in combination with alpha hydroxyacid preparations. The inclusion of a tretinoin corrective composition in the present treatment regimen may aid in keratinocyte activity regulation, mitosis, repairing damaged DNA, blood vessel formation such as angiogenesis, and the creation of a soft epidermis.

The incorporation of retinoid containing corrective composition into the sequential treatment regimen of the present disclosure can promote a smoother less wrinkled skin and can be effective in treating sun damage, wrinkling, hyperpigmentation and facial roughness. Although not wishing to be bound by this disclosure, it is believed that tretinoin passes through the skin cell membranes to the nucleus wherein it binds to nuclear receptors and regulates transcription of genes that mediate the rate of cell division and turnover, cell differentiation and formulation of new healthy collagen and the repair of elastin. As a result skin can be firmer from the collagen formation as well as more flexible from the repair of elastin.

Tretinoin also increases the formation of normal keratinocytes (cells making up about 90% of the epidermis) and fibroblasts (connective tissue cells which secrete an extracellular matrix rich in collagen and other macromolecules), decreases melanocyte activity (which offers better resistance to external injury and inflammation) and is found to improve angiogenesis (the formation of new blood vessels that increase skin circulation).

Suitable tretinoin compositions for use with the treatment regimen of the present disclosure utilize a medically prescribed tretinoin medication such as, for example, compositions commercially available from Johnson & Johnson under the RENOVA brand name. In embodiments, the retinoid containing stimulating composition used in the treatment regimen of the present disclosure is an oil-in-water emulsion, such as commercially available tretinoin creams containing 0.05% or 0.1% actives.

Another suitable corrective composition formulated to provide SPF protection while lightening freckles, age and liver spots. Suitable compositions of this type include the commercially available Obagi Nu-Derm® Sunfader, from OMP, Inc. of Long Beach, Calif., which contains a PABA-free sunscreen. Each gram of Obagi Nu-Derm® Sunfader contains hydroquinone, octinoxate, and oxybenzone 5.5% in a base of purified water, cetyl alcohol, glycerin, sodium lauryl sulfate, stearyl alcohol, tocopheryl acetate, ascorbic acid, sodium metabisulfite, disodium EDTA, methylparaben, saponins, propylparaben, BHT and butylparaben.

Another suitable commercially available corrective composition is Obagi Nu-Derm® Clear from OMP, Inc. of Long Beach, Calif. One gram of Obagi Nu-Derm® Clear contains hydroquinone in a base of purified water, cetyl alcohol, glycerin, sodium lauryl sulfate, stearyl alcohol, tocopheryl acetate, ascorbic acid, sodium metabisulfite, lactic acid, saponins, disodium EDTA, methylparaben, BHT, propylparaben and butylparaben.

Other corrective compositions include exfoliating lotion to smooth and tone rough or damaged skin. Suitable exfoliators include Obagi Nu-Derm® Exfoderm, and Obagi Nu-Derm® Exfoderm Forte. Obagi Nu-Derm® Exfoderm typically contains water, ethoxydiglycol, phytic acid, glycerin, cetearyl alcohol, glyceryl stearate. PEG-100 stearate, canola oil, isohexadecane, magnesium aluminum silicate, potassium cetyl phosphate, cetyl alcohol, bis-diglyceryl polyacyladipate-2, dimethicone, polysorbate 0, PEG-150 stearate, steareth-20, xanthan gum, glycereth-7, tocopheryl acetate, saponins, phenoxyethanol, methylparaben, propylparaben, butyparaben, ethylparaben, isobutylparaben. Obagi Nu-Derm® Exfoderm Forte typically contains purified water, glycolic acid, emulsifying wax, triethanolamine, glycerin, lactic acid, caprylic/capric triglyceride, kalaya oil, stearic acid, cetyl alcohol, dimethicone, methylparaben, propylparaben, saponins.

Stable Corrective Compositions

In any topical treatment regimen, instability of the topical composition containing the active may cause degradation of the active requiring application of unnecessarily large amounts of the active ingredient and of potentially irritating degradation by-products to the skin. It has been found that these problems can be eliminated or reduced by the use of corrective composition having three years of stability at room temperature.

Accordingly, in another aspect the present disclosure relates to stable active containing corrective compositions. These stable corrective compositions can be made by the methodology described in this disclosure; however any method of making the corrective compositions may be employed so long as they achieve the desired stability. Thus, the stable corrective compositions are formulated, manufactured and packaged in accordance with this disclosure in a manner which enables the composition to remain in the package without discoloring. As used herein the term “stable” means that the composition when in a closed container remains within the tolerances and limits set forth in US Pharmacopoeia and/or the US FDA guidelines or monographs for compositions containing any particular active ingredient or combination of active ingredients. The entire US Pharmacopoeia and collection of US FDA guidelines or monographs for compositions containing any particular active ingredient or combination of active ingredients are too voluminous to present in their entirety herein and thus are instead incorporated in their entirety by this reference. With respect to topical compositions, the tolerances and limits are frequently presented relative to the labeled amount. As one illustrative example, for hydroquinone cream, the acceptable tolerance is not less than 94.0 percent and not more than 106.0 percent of the labeled amount of C₆H₆O₂. As another illustrative example, for tretinoin cream, the acceptable tolerance is not less than 90.0 percent and not more than 130.0 percent of the labeled amount of C₂₀H₂₈O₂. Those skilled in the art will readily be able to identify the tolerances and limits for other compositions containing other active ingredients.

As those skilled in the art will appreciate, the container-liner-closure system used to store the composition will affect the stability of the active ingredient. It should be understood that a composition need not be stable in all containers to be stable in accordance with this disclosure. Stability in at least one type of container is sufficient for a composition to be stable as that term is used herein.

In embodiments, stable corrective compositions in accordance with the present disclosure can be stable for at least three years at room temperature. Stability of the present compositions can be evaluated through accelerated stability studies. In these studies, the packaged composition is maintained at an elevated temperature for a period of time after which it is examined. The exposure to elevated temperatures for a given period correlates to a correspondingly longer period of time at room temperature. Thus, for example, if a product remains within the required tolerances and limits when maintained for a period of 12 weeks at a temperature of 40° C. and 12 months further at room temperature, one can conclude that the product has a shelf life of greater than two and up to three years at room temperature. Those skilled in the art will envision other testing to confirm the stability of the products described herein. Further testing methodology is described in the working examples below.

The stable corrective compositions of the present disclosure contain an oxygen sensitive active ingredient and a unique mixture of ingredients and is prepared by initially mixing an aqueous phase with an oil phase.

The aqueous phase includes water, humectants, emulsifiers, preservatives, chelating agents, reducing agents and a pH adjuster. Purified water can advantageously be used, such as, for example, de-ionized water or USP water. As those skilled in the art will appreciate, tap water varies widely in its composition from location to location, which makes the composition of the water unreliable and inconsistent as a medium for the present compositions. Water can be treated to have substantially the same composition, irrespective of the source.

In a particularly useful water treatment, tap water is passed sequentially through a water softening reverse osmosis unit and a deionizing unit to provide pharmaceutical grade high purity water having a resistivity of one mega ohm or greater. Before entering the reverse osmosis system, the water advantageously can be passed through one or more filters and a water softener. The filters can contain various filter media, including carbon, sand, diatomaceous earth, cellulosic materials and the like. The water exiting the deionizing unit may be passed through one or more final filters, sterilized and used in the formulation. Other suitable water purification systems can be used so long as the resulting resistivity is one megaohm or greater.

Suitable preservatives for the aqueous phase include, but are not limited to methylparaben, sodium butylparaben, benzoic acid and its salts and esters, benzyl alcohol, urea derivatives such as diazolidinyl urea, imidazolidinyl urea, and DMDM hydantoin, sorbic acid and its salts, and the like. Typically, the preservatives are present in an amount from about 0.01 to about 5% by weight of the total composition. In embodiments, the preservatives are present in an amount from about 0.05 to about 1% by weight of the total composition.

Suitable chelating agents for the use in the aqueous phase include, but are not limited to edetate disodium, EDTA (ethylenediaminetetraacetic acid) and its salts, for example, trisodium NTA (nitrilotriacetic acid), etidronic acid and its salts, sodium dihydroxyethylglycinate, citric acid and its salts, and combinations thereof. Typically, the amount of chelating agent(s) is from about 0.01 to about 5% by weight of the total composition. In embodiments, the chelating agents are present in an amount of about 0.05 to about 1% by weight of the total composition.

Suitable humectants for use in the aqueous phase include, but are not limited to polyhydric alcohols including glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol and hyaluronic acid and its salts. It should, of course be understood that combinations of two or more humectants can be included in the present compositions. Typically, humectants are present in an amount from about 1 to about 20% by weight of the total composition. In embodiments, humectants are present in an amount from about 2 to about 5% by weight of the total composition.

Suitable emulsifiers for use in the aqueous phase are surfactants. Useful surfactants can be ionic or nonionic, and they can be used alone or in admixture. Illustrative examples of suitable surfactants include cetearyl alcohol and sodium cetearyl sulfate, PEG-1000 monocetyl ether, or quaternary ammonium salts such as alkyl trimethyl ammonium bromide; polyol ester glycerol monostearate and potassium stearate, sodium lauryl sulfate (SLS), and ethoxylated fatty alcohols constitute good coemulsifiers. Fatty acids like stearic acids may be included to regulate the consistency of the emulsion. Optionally, polymers such as carbomers also can be included. Particularly useful emulsifiers for use in the aqueous phase are sodium lauryl sulfate, saponins or combinations thereof. Typically, the emulsifiers are present in an amount from about 1 to about 20% by weight of the total composition. In embodiments, the emulsifiers are present in an amount from about 2 to about 5% by weight of the total composition.

The pH of the aqueous phase can be adjusted to between about 2 to 4, such that the final product has a pH between about 2 to 4. Agents suitable for adjusting the pH of the aqueous phase include, but are not limited to citric acid, phosphoric acid, lactic acid or glycolic acid. Typically, the pH adjustment agents are present in an amount from about 0.01 to about 5% by weight of the total composition. In embodiments, the pH adjustment agent is present in an amount from about 0.1 to about 1.0% by weight of the total composition.

Suitable reducing agents for use in the present stable corrective compositions include, but are not limited to ascorbic acid, propyl gallate and sulfites, including but not limited to sulfites, bisulfites, metabisulfites, their salts, and their derivatives. Sodium metabisulfite is one useful sulfite. These antioxidants can be advantageously used because they have greater tendencies to oxidize than many oxidation-sensitive active ingredients. Sodium metabisulfite has the added advantage that it does not discolor by oxidation. Typically, the reducing agents are present in amounts from about 0.1 to about 10% by weight of the total composition. In embodiments, the reducing agents are present in an amount from about 0.5 to about 5% by weight of the total composition.

The aqueous phase can be prepared by combining the various ingredients while mixing with heating (e.g., to 70-75° C.). In embodiments, the aqueous phase can be prepared by mixing the various ingredients in water that has either been heated until it boils to eliminate dissolved oxygen or treated with and an inert, oxygen-free gas (such as nitrogen) to replace the dissolved oxygen while mixing with heating (e.g., to 70-75° C.). The tank in which the aqueous phase (and, for that matter, all the compositions used in the formulation) is prepared can advantageously be made from a material that does not release metallic ions into the composition. For example, a glass tank can be employed. As another example, a 316 stainless steel tank that has been pre-treated to chelate metallic ions present therein can be used. Chelating can be achieved, for example, by contacting the interior of the tank with a sacrificial chelating acid. Suitable sacrificial chelating acids include organic acids such as citric acid and the like. In particularly useful embodiments, a stainless steel tank (of grade 316 or higher) is treated with a 10% citric acid solution heated to no higher than 40° C. prior to use. Mixing is continued under a positive nitrogen pressure greater than 1 atmosphere until all solids are dissolved. As an alternative, the previous process can be done under vacuum and the nitrogen of pressure greater than 1 atmosphere flushed into the tank. As those skilled in the art will appreciate, this and other tanks employed in making the present compositions advantageously can be kept at above atmospheric pressures so that nitrogen leaks out rather than oxygen-containing air leaking into the vessels.

The aqueous phase in then mixed with an oil phase, such as, for example, in a tank such as the tank described above made from material that does not release metallic ions into the composition. The oil phase can include emollients, preservatives and antioxidants.

Suitable emollients for use in the oil phase include cosmetically acceptable liquid oils. The cosmetically acceptable liquid oil is liquid at room temperature. The cosmetically acceptable liquid oil can be liquid hydrocarbon oil, liquid natural oil, liquid fatty alcohol, liquid fatty acid, liquid fatty acid ester, liquid silicone oil, and paste wax and mixtures thereof.

Non-limiting examples of the liquid hydrocarbons suitable for use in the oil phase include squalane, liquid mineral oil, and liquid polybutene. Non-limiting examples of the liquid natural oil derived from plants useful in the present compositions include almond oil, olive oil, sesame oil, safflower oil, avocado oil, cottonseed oil, jojoba oil, castor oil, soybean oil, palm kernel oil, coconut oil, and hydrogenated vegetable oil. Non-limiting examples of the liquid natural oil derived from animal sources useful in the present compositions include mink oil and egg yolk oil. Non-limiting examples of the liquid fatty alcohol useful in the present compositions are isostearyl alcohol, lanolin alcohol, oleyl alcohol, hexadecyl alcohol, octyidodecanol alcohol, linoleyl alcohol, linolenyl alcohol, lauryl alcohol and arachidyl alcohol. Fatty acid can be natural or synthetic, saturated, unsaturated, linear, or branched. Non-limiting examples of fatty acid useful in the present compositions are caprylic, isostearic, linoleic, ricinoleic, and oleic acid. Non-limiting examples of the liquid fatty acid ester useful in the present compositions are cetyl octanoate, glyceryl trioctanoate, isopropyl linoleate, isopropyl myristate, isopropyl oleate, ethyl laurate, ethyl linoleate, octyl dodecyl myristate, octyl palmitate, octyl isopelargonate, octyl dodecyl lactate, isotridecyl isononanoate, oleyl oleate, isostearyl myristate, neopentyl glycol dioctanoate, and di(capryl/capric acid) propylene glycol and mixtures thereof. Other suitable esters include triglycerides such as caprylic triglycerides, capric triglyceride, isostearic triglyceride and adipic triglyceride. Non-volatile, straight, and branched silicone oils such as dimethicone and phenyl dimethicone are also useful. Other cosmetically acceptable ingredients like sunscreens include octyl methoxy cinnamate, cinoxate, and 2-ethylphexyl p-dimethyaminobenzoate and the like.

Either one kind or two or more kinds of the cosmetically acceptable liquid oil can be used in the present stable corrective compositions. Particularly useful emollients include cetyl alcohol, stearyl alcohol and combinations thereof. Typically, the emollients are present in an amount from about 5 to about 25% by weight of the total composition. In embodiments, the emollients are present in an amount from about 7.0 to about 15% by weight of the total composition.

Suitable antioxidants for use in the oil phase include, but are not limited to BHT, BHA, tocopherol, tocopheryl acetate, ascorbyl palmitate, propyl gallate, and the like. Typically, the antioxidants are present in an amount from about 0.01 to about 10% by weight of the total composition. In embodiments, the antioxidants are present in an amount from about 0.1 to about 2% by weight of the total composition.

Suitable preservatives for use in the oil phase include propylparaben, isopropylparaben, butylparaben, and isobutylparaben, and the like. The preservatives in the oil phase typically are present in an amount from about 0.01 to about 5% by weight based on the total composition. In embodiments, preservatives are present in an amount from about 0.05 to about 2% by weight based in the total composition.

The oil phase can be prepared by simply adding the ingredients for the oil phase while heating (e.g., to 70°-75° C.) with moderate agitation. The oil phase is then added to the aqueous phase under an inert, oxygen-free atmosphere with moderate agitation. The batch can then be cooled to (e.g., to 500-55° C.).

In embodiments, an aqueous premix of reducing agents is prepared in a separate tank. Suitable reducing agents include, but are not limited to ascorbic acid, propyl gallate and sulfites, including but not limited to sulfites, bisulfites, metabisulfites, their salts, and their derivatives. A particularly useful reducing agent is sodium metabisulfite. Since certain active ingredients have a tendency to discolor through oxidation, these antioxidants are chosen to have greater tendencies to oxidize than the active. Sodium metabisulfite has the added advantage that it does not discolor by oxidation. Typically, the reducing agents are present between about 0.1 and about 10% by weight of the total composition. In embodiments, the reducing agents are present in an amount of about 0.5 to 5% by weight of the total composition.

The premix containing reducing agents can be prepared by adding the reducing agents to water at room temperature and mixing until all solids are dissolved. The reducing agent premix is then added to the batch containing the mixed oil and aqueous phases. The tank containing the batch is again flushed with an inert oxygen-free gas such as nitrogen and the batch is maintained under a blanket of such gas until further use.

The active ingredient that is susceptible to oxidation is then added to the batch. Where the active is water soluble, an aqueous solution can be used. Where the active is not water soluble, a non-aqueous solution can be used. Active ingredients that are prone to oxidation are known to those skilled in the art. Illustrative examples of oxygen-sensitive active ingredients include retinol, kojic acid; lactic acid; ascorbic acid and derivatives such as magnesium ascorbyl phosphate; hydroquinone; arbutin; and licorice root.

The active ingredient is present in amounts that provide a benefit to the skin of a user. While the amount of active used will depend on a number of factors including the specific active chosen and the benefit to be achieved, generally, amounts from about 0.01 to about 10% by weight of the total composition is suitable. In embodiments, the active is present in an amount from about 0.1 to about 5% by weight of the total composition. In other embodiments, an amount from about 0.1 to about 4.0% by weight of the total composition.

A solution of the active ingredient is prepared and added to the emulsion containing the reducing agents under an inert, oxygen free atmosphere with mixing. After the active is added, the batch can be cooled to 30°-40° C.

By preparing the present compositions under an inert, oxygen-free atmosphere at the relatively low temperatures described herein, the solubility of the oxygen-free gas in the composition is maximized. This is especially true where each of the aqueous compositions described herein is prepared using water having a resistance of one megaohm or greater. It is believed by using these conditions, the stability of the present compositions is increased, since the relatively saturated compositions cannot absorb ambient oxygen as readily.

The viscosity of the final composition can be from about 1,000 to about 50,000 centipoise (cps), to maintain suitable aesthetic properties, in embodiments from about 2,500 to about 15,000 cps. The specific gravity of the final composition can be from about 0.5 to about 1.5, in embodiments from about 0.95 to about 1.05.

The stability of these corrective compositions can be evaluated through accelerated stability studies. In these studies, the packaged composition is maintained at an elevated temperature for a period of time after which it is examined. The exposure to elevated temperatures for a given period correlates to a correspondingly longer period of time at room temperature. Generally most drug active ingredients decompose according to an Arrhenius first order kinetic rate. Thus, for example, a product at 20° C. is more stable than a product at 30° C. For a first order rate of decomposition this will correspond to approximately three times the rate of decomposition at 30° C. compared with 20° C. Thus, for example, if a product does not degrade when maintained for a period of 12 weeks at a temperature of 40° C. and 12 months further at room temperature, one can conclude that the product has a shelf life of greater than two and up to three years at room temperature. Those skilled in the art will envision other testing to confirm the shelf life of the products described herein.

Thus, certain embodiments of the stable corrective compositions in accordance with this disclosure have the compositions set forth in Table 1.

TABLE 1

| | | % by weight | Quantity used to | |
|---|---|---|---|---|
| | % of total | of the total | produce 1,500 kg | |
| Compound | composition | composition | batch | |
| Purified water | 70-85 | 67.42 | 1011.3 | kilograms |
| Preservatives | .01-1.5 | 0.23 | 3.45 | kilograms |
| Chelating Agent | .01-0.5 | 0.30 | 5.0 | kilograms |
| Humectants | 1-10 | 6.00 | 90.0 | kilograms |
| Anionic | .01-5 | 1.00 | 15.0 | kilograms |
| Surfactants | | | | |
| Nonionic | .01-5 | 0.5 | 7.5 | kilograms |
| Surfactants | | | | |
| Organic Acid | .01-5 | 1.0 | 15 | kilograms |
| C12-C18 Alkyl | 2-50 | 9.80 | 147 | kilograms |
| Alcohols | | | | |
| Antioxidants | .01-10 | 2.55 | 30.75 | kilograms |
| Reducing Agents | .01-5 | 0.7 | 10.5 | kilograms |
| Active Ingredient | .01-10 | 5.00 | 75.0 | kilograms |
| Emollient | 1-10 | 6 | 90 | kilograms |

The composition can be packaged in suitable containers such as tubes or bottles. Suitable containers are commercially available from a variety of suppliers. A wide variety of containers and suppliers are listed in the CPC Packaging Directory. (See, Buyers' Guide under “Containers” at www.cpcpkg.com.)

It has been found that the selection of a container material less susceptible to the permeation of oxygen through its walls promotes stability of the compositions placed therein. Accordingly, the selection of a container material less susceptible to the permeation of oxygen also inhibits discoloration and oxidation of compositions therein. Table 2 below shows some diffusion figures of gaseous permeants in high density poly ethylene (HDPE) and low density poly ethylene (LDPE).

TABLE 2
Gas Diffusion Figures

| Experimental | Temperature: | 0 degrees Celsius (273 |
|---|---|---|
| Conditions: | | Kelvin) |
| | Pressure: | 1 bar |
| | | (1 × 10⁵ Pa) |
| | Activity of permeant: | 1 |

| | | | | Swelling | Permeability* | |
|---|---|---|---|---|---|---|
| Polymer | Permeant | Solubility | Diffusivity | Factor | (m³ₛₜₚm⁻³ bar⁻¹) | Permeability* |
| (—) | (—) | m³ₛₜₚm⁻³bar⁻¹ | m²s⁻¹ | α: (—) | m²s⁻¹ | gram m⁻¹s⁻¹bar⁻¹ |
| HDPE (High | CO2 | | | — | 3.5 × 10⁻¹² | 7.4 × 10⁻⁹ |
| Density Poly | O2 | | | — | 7.8 × 10⁻¹³ | 1.1 × 10⁻⁹ |
| Ethylene) | N2 | | | — | 2.3 × 10⁻¹³ | 2.8 × 10⁻¹⁰ |
| | H2 | | | — | 3 × 10⁻¹³ | 3 × 10⁻¹¹ |
| LDPE (Low Density | CO2 | 0.25 | 3.7 × 10⁻¹¹ | — | 9.4 × 10⁻¹² | 2.0 × 10⁻⁸ |
| Poly Ethylene) | O2 | 0.05 | 4.6 × 10⁻¹¹ | — | 2.3 × 10⁻¹² | 3.2 × 10⁻⁸ |
| | N2 | 0.02 | 3.2 × 10⁻¹¹ | — | 6.4 × 10⁻¹³ | 7.9 × 10⁻¹⁰ |
| | H2 | 0.16 | 4.7 × 10⁻¹¹ | — | 7.6 × 10⁻¹² | 6.7 × 10⁻¹⁰ |

Note:
figures are applicable at 273 Kelvin and in the pressure of 0.1-˜3 Bar.
*Based on stationary/steady state. Calculate mass transfer per square meter (gram m⁻² bar⁻¹ s⁻¹) by dividing permeability mass figure (gram m⁻¹ bar⁻¹ s⁻¹) with thickness of wall (m).

As shown in Table 2 the oxygen transfer rate is approximately 3 times faster for LDPE, therefore any blend of LDPE will be detrimental to the shelf life of the compositions containing active ingredients susceptible to oxidation. Compositions formulated in accordance with this disclosure having a 3 year shelf life placed in a pure HDPE container would have a decreased shelf life when placed in an entirely LDPE container where only about 12 months stability would be expected. Moreover, compositions having a three year shelf life would expect about 24 months stability when placed in a 50/50 blend (HDPE/LDPE) container.

Accordingly suitable containers for packaging the compositions of the present disclosure include any container that minimizes oxygen transfer through the walls of the container. In some embodiments the oxygen transfer rate of the material is less than 1.1×10⁻⁹ gram m⁻¹s⁻¹bar⁻¹. Examples of suitable high density containers include, but are not limited to high density poly ethylene and high performance polyethylene (HPPE).

In embodiments, the stable corrective composition is packaged in accordance with this disclosure in a manner that maintains the stability of the product over time. A cylinder of inert, oxygen-free gas (such as nitrogen) is attached with a flow meter is secured at the beginning of the filler line. A tube line is connected from the flow meter to a sensor attached to a conveyor line. The flow meter is capable of flushing a desired volume of inert, oxygen-free gas per hour. To ensure ample amount of inert, oxygen-free gas per container, three trial flush runs on empty containers are conducted prior to start of filling as part of the set-up.

A liquid hopper is blanketed with inert, oxygen-free gas and liquid is pumped through a filler line. The containers are manually placed into a holder to level each container and subjected to at least half a second inverted burst of inert gas prior to filling. The containers are then filled with stable corrective composition. The containers move down the conveyor after filling. The containers are then capped, either manually or by machine.

Treatment regimen in accordance with this disclosure can be more effective in achieving skin penetration of an active ingredient compared to simple application of the active alone. In embodiments, a treatment regimen in accordance with this disclosure thus includes the sequential use of three products; namely, a cleanser or foaming gel, a toner and a mixture of the compositions previously described herein (referred to hereinafter as “a blending composition”) and an active agent.

Non-limiting examples of categories of active ingredients that can be mixed with the blending composition include one or more of sunscreens, anti-wrinkle actives, anti-microbials, anti-acne actives, anti-psoriasis actives, anti-eczema actives, topical anesthetic actives, anti inflammatory actives, vitamin actives, whitening actives, and the like.

Sunscreens which optionally may be combined with the blending composition include without limitation those selected from the group comprising amino benzoic acid and derivatives, such as para-amino benzoic acid (PABA), glyceryl-PABA (Lisadimate), Padimate O, Roxadimate; anthrinalates, including methylanthrynilate; benzophenones, including dioxybenzone, oxybenzone and sulisobenzone, 3-benzophenone (Uvinul M40) 4-N,N- dimethylaminobenzoic acid ester with 2,4-dihydroxybenzophenone; camphor derivatives including 3-(4-methylbenzylidene) camphor, 3-benzylidene camphor; cinnamates including DEA-p-methoxycinnamate, ethyl-hexyl p-methoxy cinnamate, octocrylene, octyl methoxy cinnamate (Parasol MCX); dibenzoyl methanes including butylmethoxydibenzoylmethane (Parsol 1789), salicylates including, homomenthyl salicylate, octyl salicylate, trolamine methyl salicylate; metal oxides including titanium dioxide, zinc oxide and iron oxide; 2-phenylbenzimidazole-5-sulfonic acid; 4,4-methoxy-t-butyldibenzoylmethane; and mixtures thereof.

Anti-wrinkle and anti-aging actives which may be combined with the blending composition include without limitation hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); and mixtures of these acids. Further anti-wrinkle agents include retinoic acid, gamma-linolenic acid; fruit acids, sugar cane extract and glycomer in cross-linked alpha nutrium; and mixtures thereof. Skin peel agents for example phenol, phytic acid, trichloroacetic and acetic acid may also be used in accordance with the present disclosure. Salicylic acid, lactic acid, trichloroacetic and glycolic acid are suitable for use herein.

Sunless tanning actives which may be combined with the blending composition include without limitation dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof.

Antimicrobials which may be combined with the blending composition include all antibiotics, antimicrobial agents and antimicrobial peptides. Antibiotics that may be used include inter alia dermatologically acceptable salts of tetracycline and tetracycline derivatives, gentamycin, kanamycin, streptomycin, neomycin, capreomycin, lineomycin, paromomycin, tobramycin, erythromycin, triclosan, octopirox, parachlorometa xylenol nystatin, tolnaftate, miconazole hydrochloride, chlorhexidine gluconate, chlorhexidine hydrochloride, methanamine hippurate, methanamine mandelate, minocycline hydrochloride, clindamycin, cleocin, b-lactam derivatives such as aminopenicillin and mixtures thereof. Suitable for use herein are chlorhexidine gluconate and triclosan.

Anti microbial agents that may be used in accordance with the present disclosure include for example benzoyl peroxide and salicylic acid.

Antimicrobial peptides useful herein are for example magainin, nicin and cecropin.

Anti-acne actives which may be combined with the blending composition include without limitation keratolytic agents including lactic acid, pyruvic acid, salicylic acids, urea and N-acetylcysteine; retinoids, and retinoid analogs such as tretinoin, cis and trans retinoic acid, retinol and retinol palmitate, isotretinoin-13-cis-retinoic acid; antibiotics and antimicrobial agents such as tetracycline, erythromycin, minocycline, clindamycin, trimethoprim-sulphamethazole and anti-microbial peptides (nicin, for example); steroids, such as hydrocortisone; gamma-linolenic acid and mixtures thereof. Further anti-acne actives that may be used include without limitation benzoyl peroxide; alpha and beta hydroxy acids; sulfacetamide and sulfur and mixtures thereof. Particularly useful anti-actives are salicylic acid, benzoyl peroxide and retinoids.

Anti-psoriasis actives for use in the present disclosure include without limitation salicylic acid; mometasone furoate; steroids including corticosteroids such as cortisone and oluxclobetasol propionate; 5-fluorouracil; epinephrine; anthralin; vitamin D3 analogs, such as calcipotriene; methotrexate; masprocol; trimethaxate gluconate; retinoids; cyclosporin; paclitaxel; 5-amino levulinic acid; bergasol; tin-ethyl etio purpurin; benzoporphyrin derivatives; antibodies, such as ABX-IL8 antibody, CD11a monoclonal antibody and ICM3 monoclonal antibody; enzyme inhibitors, including tryptase inhibitor and phospholipase A-2 inhibitors; angiogenesis blocking agents; T-cell blocking agents and mixtures thereof. (h)

Anti-eczema actives useful herein include urea; evening primrose oil; plant extracts; hydrocortisone; an immunomodulator; tar combined with fatty acids obtained from banana; and mixtures thereof.

Topical anesthetic actives which may be combined with the blending composition include tetracaine, lidocaine, editocaine, bupivacaine, pramoxine; and mixtures thereof.

Anti inflammatory actives which may be combined with the blending composition include steroidal actives such as hydrocortisone as well as non-steroidal actives including propionic derivatives; acetic acid derivatives; biphenylcarboxylic acid derivatives; fenamic acid derivatives; and oxicams. Examples of anti-inflammatory actives include without limitation acetaminophen, oxaprozin, pranoprofen, benoxaprofen, bucloxic acid, elocon; and mixtures thereof.

Vitamin actives which may be combined with the blending composition include vitamin A and derivatives, including retinoic acid, retinyl aldehyde, retin A, retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin E linolate and vitamin E acetate; vitamin K and derivatives; vitamin Q (ubiquinone) and mixtures thereof.

One class of actives which may be combined with the blending composition are proteins and peptides. In principle, any desired protein or peptide may be produced using this technology and oil bodies comprising these recombinant proteins may be incorporated in the emulsions of the present disclosure. Proteins and peptides which may be used in accordance with the present disclosure include enzymes such as proteases (e.g. bromelain, papain, collagenase, elastase), lipases (e.g. phospholipase C), esterases, glucosidases, exfoliating enzymes; antibodies and antibody derived actives, such monoclonal antibodies, polyclonal antibodies, single chain antibodies and the like; reductases; oxidases; peptide hormones; natural structural skin proteins, such as elastin, collagen, reticulin and the like; growth factors such as platelet derived growth factor (PDGF) and epidermis derived growth factor (EGF); anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments, derivatives of these proteins and peptides; and mixtures thereof.

Further active ingredients which may be combined with the blending composition include a amino acid and amino acid derivative; insect repellants; fungicides (such as ketoconazole); anti-viral agents (such as acyclovir); anti-cancer agents; plant extracts; anti-hemorrhoid compounds; anti-dandruff compounds; hair loss stimulating compound; a nucleic acid (DNA, RNA and derivatives); anti-scabies agent (such as permethrin); anti-wart agents (such as podophyllotoxin); and mixtures thereof.

In embodiments, stable corrective composition is formulated to contain hydroquinone combined with a preservative; a chelating agent; an emulsifier; a humectant; a pH adjuster; an antioxidant; an emollient; a reducing agent and water. The formulation remained stable for three years at room temperature.

Radiofrequency Cosmetic Techniques

The second step in the treatment process is performing a radiofrequency procedure on preconditioned skin. Radiofrequency procedures are used clinically for cosmetic and therapeutic purposes. Such procedures target the lower layers of skin such as the dermis, while leaving the skin's surface (epidermis) unharmed and intact. As used herein, radiofrequency procedure refers to any procedure that uses electromagnetic energy to heat tissue. Such procedures pass radiofrequency electricity through the skin to heat up tissue. This can cause tissue to contract and promote collagen formation. Radiofrequency procedures are considered non-ablative and performed using instruments and techniques that can injure the dermis and/or dermal vasculature to stimulate wound repair response, fibroblast stimulation, and/or collagen formation.

Radiofrequency procedures for use in accordance with the present disclosure include any radiofrequency rejuvenation procedures or resurfacing technique that can improve the appearance of skin. Such radiofrequency procedures can be varied as known in the art by such factors as the patient's skin type, rejuvenation goals, recovery time priorities, threshold for complications, and esthetic expectations. All techniques can be performed in accordance with techniques known in the art by a physician.

One non-limiting example of a radiofrequency procedure is the Thermage ThermaCool™ system designed by the Thermage Corporation of Hayward, Calif. A description of its products and technology overview are listed on the company's webpage. (See, “Products” and “For Physicians” at http://www.thermage.com/index.cfm). This procedure uses a radiofrequency device having 330-watt, 6-megahertz high frequency generator that employs capacitive coupling technology within a tip device for delivering radiofrequency energy deep into dermal tissues. The device is characterized as monopolar and can heat tissue to tighten skin and renew facial contours. The radiofrequency device works by providing a capacitive coupling electrode which disperses radiofrequency energy to create an electric field across the surface of the tip and into the tissue. With a frequency of 6 megahertz, the electric field changes polarity 6 million times per second. In response to the rapid change in polarity, charged particles in the skin change orientation at the same frequency, causing skin tissue to heat. Moreover the device provides contact cooling which cools the epidermis and upper dermal layers. This procedure can be performed in accordance with techniques known in the art by a physician.

Post-Treatment

Optionally, the treatment regimen includes post-treating the pre-conditioned/treated skin. Typically, the type of radiofrequency procedure performed on the skin treatment area will dictate the type of post-treatment compositions to be applied. For example, the procedure can be varied depending on the apparatus used by the medical personnel in performing the procedure. Furthermore, the post-treatment can include repeating the pre-treatment steps described above with the same or different pre-treatment compositions including any supplementary compositions and corrective compositions described above.

It should be noted that post-treatment can comprise not just a single application of a single corrective or supplementary composition but can be a sequentially applied treatment. For example, multiple supplementary compositions can be used as well as multiple corrective compositions. Thus the classification as a post-treatment composition is not intended to limit the post-treatment compositions in any way to only those post treatment compositions mentioned herein.

In embodiments, after treating the preconditioned skin with a radiofrequency procedure, skin is post-treated by another sequential topical application of one or more corrective compositions, and one or more supplementary compositions in a morning regimen; followed by the sequential topical application of one or more corrective compositions, including tretinoin, and one or more supplementary compositions in an evening regimen. Post-treatment of preconditioned skin enhances the benefits of the radiofrequency procedure by extending the duration of the cosmetic benefit received as well limiting adverse events associated with radiofrequency procedures.

In embodiments, the first post-treatment occurs in the morning hours and includes the application of an effective amount of one or more preparatory compositions (e.g., gentle cleanser, and toner) followed by application of an effective amount of one or more corrective compositions (e.g., Obagi Nu-Derm® Exfoderm, and/or one or more hydroquinone compositions such as Obagi Nu-Derm® Clear) followed by the application of an effective amount of one or more protective compositions (e.g., Obagi Nu-Derm® UV Physical block). Note that where a procedure has caused injury or removal of the epidermis, the application of exfoliant can be delayed until skin has re-epithelialized (or redness has greatly subsided). In this embodiment, the second treatment occurs in the evening hours and includes the topical application of an effective amount of one or more preparatory compositions (e.g., gentle cleanser, and toner) followed by application of an effective amount of one or more corrective compositions (e.g., hydroquinone and tretinoin compositions). In embodiments, tretinoin is used either alone or in combination with other corrective compositions during the evening treatment.

Kit Components

As the pre-procedure treatment regimen requires the sequential application of various components, it has also been found that kits greatly facilitate the user in performing the pre-treatment regimen consistently. Such kits can include:

Foaming Gel
Toner
Obagi Nu-Derm ® Clear
Obagi Nu-Derm ® Exfoderm Forte
Blender
Healthy Skin Protection SPF 35
Tretinoin (0.1% or 0.05%)

As the treatment regimen requires the sequential application of various post procedure components, it has also been found that kits greatly facilitate the user in performing the post-treatment regimen consistently. Such kits can include:

Gentle Cleanser
Toner
Obagi Nu-Derm ® Clear
Obagi Nu-Derm ® Exfoderm
Blender
UV Physical Sunblock
Tretinoin (0.1% or 0.05%

The corrective and supplemental compositions of the pre-procedure treatment regimen may be applied as follows:

| | | First | Second |
|---|---|---|---|
| Pre-Treatment | | Appli- | Appli- |
| Regimen for RF | | cation | cation |
| Procedure | Product | (a.m.) | (p.m.) |
| Apply nickel size | Foaming Gel | X | X |
| amount of preparatory | Toner | X | X |
| composition to face | | | |
| and neck, then wash | | | |
| off. | | | |
| Apply large pea size | Obagi Nu- | X | X |
| amount of corrective | Derm ® Clear | | |
| composition to face | Nu-Derm ® | X | |
| and neck. | Exfoderm Forte | | |
| | Obagi Nu- | | X |
| | Derm ® Blender | | |
| | Tretinoin | | X |
| Apply pea size | Sun screen | X | |
| amount of protective | or sun block | | |
| composition to face | | | |
| and neck. | | | |

The corrective and supplemental compositions of the post-treatment regimen may be applied as follows:

| Post-treatment | | First | Second |
|---|---|---|---|
| Regimen for RF | | Application | Application |
| Procedure | Product | (a.m.) | (p.m.) |
| Apply pea size | Cleanser | X | X |
| amount of preparatory | Toner | X | X |
| composition to face | | | |
| and neck, then wash | | | |
| off. | | | |
| Apply large pea size | Obagi Nu- | X | X |
| amount of corrective | Derm ® Clear | | |
| composition to face | Obagi Nu- | X | |
| and neck. | Derm ® Exfoderm | | |
| | Obagi Nu- | | X |
| | Derm ® Blender | | |
| | Tretinoin | | X |
| Apply pea size | Physical | X | |
| amount of protective | UV Block | | |
| composition to face | | | |
| and neck. Reapply as | | | |
| needed. | | | |

These instructions are illustrative. Those skilled in the art may readily envision other instructions. The second application may be performed at least four hours after the first treatment for both the pre-treatment and the post-treatment.

In embodiments, a patient follows a prescribed treatment regimen twice a day (in the morning and at night) for up to about nine weeks prior to the radiofrequency procedure, preferably from about one to about seven weeks prior to the radiofrequency procedure, most preferably from about three to about four weeks prior to the radiofrequency procedure. The pre-treatment regimen involves applying designated products from the commercially available Obagi Nu-Derm® system and/or prescription product in the smallest possible amount sufficient to cover the site intended for the radiofrequency procedure, in embodiments, the entire face of the patient even if only a small area of the face is to receive the radiofrequency treatment. The regimen may advantageously be as follows:

| | Morning | Evening |
|---|---|---|
| Prepare | Foaming Gel | Foaming Gel |
| | Toner | Toner |
| Correct | Clear 4% Hydroquinone 1 | Clear 4% Hydroquinone 1 gm |
| | gm | |
| Stimulate | | Tretinoin 0.05% |
| | | Blender 4% HQ 0.5 gm |
| Protect | Sunfader | |

After the desired pre-treatment period, the radiofrequency procedure is performed. After the radiofrequency procedure is performed, the patient resumes treatment with the previously used treatment regimen for a post-treatment time of up to about eleven weeks, preferably about one to about nine weeks, most preferably from about four to about six weeks.

Benefits of Pre-Treatment and Optional Post-treatment

The use of the presently described methods may provide one or more benefits to the skin of the user undergoing a radiofrequency procedure. For example, by employing the methods described herein, a patient undergoing a radiofrequency procedure may observe perioral fine wrinkle improvement, periocular fine wrinkle improvement, hyperpigmentation improvement, hypopigmentation improvement, tactile roughness improvement, sallowness improvement, acne scarring improvement and/or increased overall skin quality. Additionally, a patient undergoing a radiofrequency procedure employing the methods described herein may observe no worsening of Erythema.

In order that those skilled in the art may be better able to practice the compositions and methods described herein, the following non-limiting examples are given for illustration purposes.

### EXAMPLE 1

49 year old white female in good general health is presented to dermatologist complaining of mild photoaging or wrinkled skin on face. The patient has little sun damage and indicates the desire to minimize, reduce, or eliminate photoaging and wrinkles. Dermatologist recommends radiofrequency treatment to obtain the desired cosmetic benefit of reducing photoaging and pulling out wrinkles.

Pre-treatment:

Patient is started on pre-treatment protocol to precondition skin for upcoming radiofrequency procedure. Preconditioning is intended to extend the duration of the cosmetic benefit received, as well as decreases the frequency of repeated radiofrequency treatments to obtain the desired cosmetic benefit of minimizing, eliminating, or reducing the photoaging and wrinkles.

Patient is prescribed tretinoin (0.05% or 0.1%) and provided with a pre-treatment kit containing a container of foaming gel, toner, Obagi Nu-Derm® Clear formulation, Obagi Nu-Derm® Exfoderm Forte, and Obagi Healthy Skin Protection having SPF 35, blender, and prescribed tretinoin. Each container provides enough formulation in an amount sufficient to be applied to face as instructed below for between 3 to 6 weeks prior to the radiofrequency procedure.

Each kit contains instructions for the patient to apply the pre-treatment compositions every morning. The instructions require the following steps to be followed in the morning in sequential order: 1) apply a nickel-sized amount of foaming gel to wet skin, massage into entire face and neck and rinse thoroughly; 2) apply toner using cotton pads or fingertips to entire face; 3) apply 0.5 grams of Obagi Nu-Derm® Clear to face; 4) apply Obagi Nu-Derm® Exfoderm Forte to entire face using caution in eye area because stinging may occur, and to rub in thoroughly; and 5) apply Healthy Skin Protection (SPF 35) to face and neck (Application of protective composition can be repeated after 2 hours if patient is in direct sunlight).

The instructions further require the following steps to be followed in the evening in sequential order: 1) apply a nickel-sized amount of foaming gel to wet skin, massage into entire face and neck and rinse thoroughly; 2) apply toner using cotton pads or fingertips to entire face; 3) apply 0.5 grams of Obagi Nu-Derm® Clear to face; 4) apply blender (0.5 grams) and tretinoin (0.5 grams). Apply in the evening after Obagi Nu-Derm Clear by measuring 0.5 grams of Blender, followed by a prescribed amount of tretinoin. Combine and apply evenly on entire face, extending to the hairline. Apply around eye area as directed. Note: Avoid excessive application around the corners of lips and creases around the nose.

Patient performs pre-treatment regimen in accordance with these instructions and preconditions the surface of skin in need of a radiofrequency procedure for three weeks prior to radiofrequency procedure.

Treatment:

Dermatologist performs radiofrequency procedure known as Thermage treatment designed by the Thermage Corporation of Hayward, Calif. on patient by subjecting patient's skin to radiofrequency energy. This method is performed by procedures known in the art. In state-of-the-art treatments only subtle results are achieved by one treatment, multiple treatments such as 6 or more are necessary. Here, as skin is preconditioned prior to the radiofrequency cosmetic procedure, the number of treatments needed to eliminate or minimize the photoaging and wrinkles to the desired amount is likely to be minimized or reduced.

Post-treatment:

Patient is started on post-treatment protocol to ensure that skin heals quickly, and to reduce the likelihood of post-procedural reactions or complications like post-inflammatory hyperpigmentation, erythema (redness), acne, bumps, and/or scarring. Post-conditioning likely extends the duration of the cosmetic benefit received.

Patient is prescribed tretinoin (0.05% or 0.1%) and provided with a post-treatment kit containing a container of gentle cleanser, toner, Obagi Nu-Derm® Clear formulation, Obagi Nu-Derm® Exfoderm formulation, and Obagi Nu-Derm® UV Physical Sunblock, blender, and prescribed tretinoin. Each container provides enough formulation in an amount sufficient to be applied to face as instructed below for between 3 to 6 weeks after the procedure.

Each kit contains instructions for the patient to apply the post-treatment compositions every morning. The instructions require the following steps to be followed in the morning in sequential order: 1) apply gentle cleanser to face, rinse with lukewarm water; 2) apply toner using fingertips to entire face (do not rinse); 3) apply 0.5 grams of Obagi Nu-Derm® Clear to face in feathering motion; 4) apply Obagi Nu-Derm® Exfoderm to entire face; 5) apply Physical UV Block to face and neck. Application of protective composition can be repeated after 2 hours if patient is in direct sunlight.

Instructions further require the following steps to be followed in the evening in sequential order: 1) apply a nickel-sized amount of gentle cleanser to wet skin, massage into entire face and neck and rinse thoroughly; 2) apply toner using cotton pads or fingertips to entire face; 3) apply 0.5 grams of Obagi Nu-Derm® Clear to face; 4) apply blender (0.5 grams) and tretinoin (0.5 grams). Apply in the evening after Obagi Nu-Derm® Clear by measuring 0.5 grams of Blender, followed by a prescribed amount of tretinoin. Combine and apply evenly on entire face, extending to the hairline. Apply around eye area as directed. Note: Avoid excessive application around the corners of lips and creases around the nose.

Patient performs post-treatment regimen in accordance with these instructions and post-conditions the surface of skin subjected to a radiofrequency procedure for three weeks after radiofrequency procedure. Patient does not have adverse events such as reactions or complications like post-inflammatory hyperpigmentation, erythema (redness), acne, bumps, and scarring. Wrinkles and photoaging characteristics are not likely to return during the three week post-conditioning.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A method comprising:
preconditioning an area of skin of a subject intended to receive a radiofrequency procedure by sequential topical application of a preparatory composition and a corrective composition to the area of skin.

2. A method as in claim 1 further comprising applying a protective composition after application of the corrective composition.

3. A method as in claim 1 further comprising waiting a predetermined period of time and repeating the preconditioning.

4. A method as in claim 1 further comprising performing a radiofrequency procedure on the preconditioned area of skin.

5. A method comprising:
post-treating an area of skin of a subject that has previously been treated with a radiofrequency procedure by sequential topical application of a preparatory composition and a corrective composition to the area of skin.

6. A method as in claim 5 further comprising applying a protective composition after application of the corrective composition.

7. A method as in claim 5 further comprising waiting a predetermined period of time and repeating the post-treating.

8. A method comprising:
preconditioning an area of skin of the subject intended to receive a radiofrequency procedure by administering to the area of skin a first treatment and, after a predetermined period of time, a second treatment,
the first treatment comprising the sequential topical application of one or more preparatory compositions, a first corrective composition, and one or more protective compositions;
the second treatment comprising the sequential topical application of one or more preparatory compositions, and a second corrective composition,
wherein the second corrective composition comprises a first active ingredient sensitive to oxidation and a second active ingredient comprising a retinoid.

9. The method of claim 8 wherein the one or more preparatory compositions are selected from the group consisting of cleansers, toners and combinations thereof.

10. The method of claim 8 wherein the one or more protective compositions are selected from the group consisting of sunscreens, sun blocks, moisturizers and combinations thereof.

11. The method of claim 8 wherein the first corrective composition comprises an active ingredient that is sensitive to oxidation; a preservative; a chelating agent; an emulsifier; a humectant; a pH adjuster; an antioxidant; an emollient; a reducing agent and water.

12. The method of claim 8 wherein the first corrective composition comprises hydroquinone.

13. The method of claim 8 wherein the second corrective composition further comprises a preservative; a chelating agent; an emulsifier; a humectant; a pH adjuster; an antioxidant; an emollient; a reducing agent and water.

14. The method of claim 8 wherein the first active ingredient is hydroquinone.

15. The method of claim 8 further comprising the step of waiting at least four hours between the first treatment and the second treatment.

16. The method of claim 8 further comprising treating the skin with a radiofrequency procedure.

17. The method of claim 16 further comprising post-treating the skin of the subject treated with a radiofrequency procedure by administering to the area of skin a first treatment and, after a predetermined period of time, a second treatment, the first treatment comprising the sequential topical application of one or more preparatory compositions, a first corrective composition, and one or more protective compositions;
the second treatment comprising the sequential topical application of one or more preparatory compositions, and a second corrective composition, wherein the second corrective composition comprises a first active ingredient sensitive to oxidation and a second active ingredient comprising tretinoin.

18. A method comprising
preconditioning an area of skin of the subject intended to receive a radiofrequency procedure by administering to the area of skin a first treatment and, after a predetermined period of time, a second treatment,
the first treatment comprising the sequential topical application of a cleanser, a toner, a first corrective composition containing hydroquinone, and a protective composition;
the second treatment comprising the sequential topical application of a cleanser, a toner, a second corrective composition comprising hydroquinone and a retinoid; and
performing a radiofrequency procedure on the preconditioned area of skin.

19. The method of claim 18 further comprising post-treating the area of skin subjected to the radiofrequency procedure by administering to the area of skin a first treatment and, after a predetermined period of time, a second treatment,
the first treatment comprising sequential topical application of a cleanser, a toner, a first corrective composition containing hydroquinone, and a protective composition;
the second treatment comprising sequential topical application of a cleanser, a toner, a second corrective composition comprising hydroquinone and a retinoid.

20. The method of claim 18 wherein the first pre-conditioning treatment occurs in the morning.

21. The method of claim 20 wherein second pre-conditioning treatment occurs at least four hours after the first pre-conditioning treatment.

22. The method of claim 19 wherein the first post-treating treatment occurs in the morning.

23. The method of claim 22 wherein second post-treating treatment occurs at least four hours after the first post-treating treatment.

24. A skin treatment kit for use prior to a radiofrequency procedure comprising:
a corrective composition;
at least one supplemental composition selected from the group consisting of preparatory compositions, protective compositions, and combinations thereof;
and instructions for pre-conditioning an area of skin by the ordered application of the corrective composition and the at least one supplemental compositions in preparation for a radiofrequency procedure.

25. A kit as in claim 24 wherein the corrective composition comprises:
an active ingredient that is sensitive to oxidation; a preservative; a chelating agent; an emulsifier; a humectant; a pH adjuster; an antioxidant; an emollient; a reducing agent and water

26. A kit as in claim 24 wherein the at least one supplemental composition is a preparatory composition selected from the group consisting of cleansers, toners and combinations thereof.

27. A kit as in claim 24 wherein the at least one supplemental composition is a protective composition selected from the group consisting of sunscreens, sun blocks and combinations thereof.

28. A kit comprising:
a preparatory composition;
a corrective composition;
a protective composition; and
instructions for pre-conditioning an area of skin in preparation for a radiofrequency procedure by administering to the area of skin a first treatment and, after a predetermined period of time, a second treatment,
the first treatment comprising the sequential topical application of the preparatory composition, the corrective composition, and the protective composition;
the second treatment comprising the sequential topical application of the preparatory composition, and the corrective composition.

29. A skin treatment kit for use after a radiofrequency procedure comprising:
a corrective composition;
at least one supplemental composition selected from the group consisting of preparatory compositions, protective compositions, and combinations thereof;
and instructions for post-treating an area of skin by the ordered application of the corrective composition and the at least one supplemental compositions after a radiofrequency procedure.
